(19) United States
(12) Patent Application Publication
(10) Pub. No.: US 2019/0211044 A1
(43) Pub. Date: Jul. 11, 2019

(54) Adjuvant Chemicals that Prevent Drug Tolerance and Persister Formation by Bacteria

(71) Applicant: LifeUnit Inc., Syracuse, NY (US)

(72) Inventors: Yan-Yeung Luk, Jamesville, NY (US); Pankaj Dinkar Patil, Syracuse, NY (US)

(21) Appl. No.: 16/240,527

(22) Filed: Jan. 4, 2019

Related U.S. Application Data
(60) Provisional application No. 62/613,967, filed on Jan. 5, 2018.

(51) Int. Cl. C07H 15/04; A61P 31/04

(52) U.S. Cl. CPC C07H 15/04; A61P 31/04

(57) ABSTRACT

Disclosed in certain embodiments is a compound of Formula I:

X—Z (I)

wherein X is a monosugar or disugar moiety and Z is a C₈₋₂₀ straight chain alkyl, alkenyl or alkynyl having 1-5 substituents (Y) on the first 6 carbons proximal to the disugar moiety, wherein each Y is independently C₁₋₈ linear alkyl, C₃₋₈ branched alkyl, C₃₋₈ cycloalkyl, halogen, hydroxyl, monocyclic aromatic, monocyclic heteroaromatic, bicyclic aromatic, bicyclic heteroaromatic, tricyclic aromatic, or tricyclic heteroaromatic, wherein each Y is independently optionally substituted with C₁₋₈ linear alkyl, C₃₋₈ branched alkyl, C₃₋₈ cycloalkyl, halogen or hydroxyl and pharmaceutical compositions and methods thereof.

The present invention relates to the field of pharmaceuticals for antibiotic treatment. Specifically, the present invention relates to monosugar or disugar compounds for the treatment of infections and the prevention or inhibition of biofilm formation.

### BACKGROUND OF THE INVENTION

Antibiotics that kill bacteria can also cause responses that, over time, lead to the development of antibiotic-resistant bacteria, making the treatment of infectious diseases more challenging. At sub-lethal concentrations, antibiotics can readily increase the population of tolerant bacteria; these tolerant populations require a higher antibiotic dosage to kill than typical susceptible bacteria. Studies also suggest that drug-tolerant bacteria have a higher propensity to evolve into drug-resistant bacteria, making antibiotics ineffective, even at a high dosage. Antibiotics can also enhance a pre-existing population of bacteria, called persisters, which are non-growing and thus extremely difficult to eradicate using current antibiotics.

There is a need in the art to develop an antibiotic treatment to which bacteria is less likely to develop tolerance and resistance and that is effective in the inhibition of biofilms. The antibiotic treatment should be stable and effective in treating infections.

### OBJECTS AND SUMMARY OF THE INVENTION

It is an object of certain embodiments of the present invention to provide an antibiotic pharmaceutical composition able to kill bacteria under maximal growing conditions.

It is an object of certain embodiments of the present invention to provide an antibiotic pharmaceutical composition able to inhibit bacteria growth and/or inhibit biofilm formation.

It is an object of certain embodiments of the present invention to provide an antibiotic pharmaceutical composition that inhibits biofilm and swarming activities.

It is an object of certain embodiments of the present invention to provide an antibiotic pharmaceutical composition that reduces bacteria's tolerance towards other antibiotics.

It is an object of certain embodiments of the present invention to provide an antibiotic pharmaceutical composition that prevents other antibiotics from causing persister formation from bacteria during treatment.

The above objects of the present invention and others may be achieved by the present invention which in certain embodiments is directed to a compound of Formula I:

X—Z (I)

wherein X is a monosugar or disugar moiety and Z is a C₈₋₂₀ straight chain alkyl, alkenyl or alkynyl having 1-5 substituents (Y) on the first 6 carbons proximal to the disugar moiety, wherein each Y is independently C₁₋₈ linear alkyl, C₃₋₈ branched alkyl, C₃₋₈ cycloalkyl, halogen, hydroxyl, monocyclic aromatic, monocyclic heteroaromatic, bicyclic aromatic, bicyclic heteroaromatic, tricyclic aromatic, or tricyclic heteroaromatic, wherein each Y is independently optionally substituted with C₁₋₈ linear alkyl, C₃₋₈ branched alkyl, C₃₋₈ cycloalkyl, halogen or hydroxyl.

In other embodiments, the present invention is directed to a compound of Formula I:

X—Z (I)

wherein X is a monosugar or disugar moiety and Z is a C₈₋₂₀ straight chain alkyl, alkenyl or alkynyl having 2-5 substituents (Y) on the first 6 carbons proximal to the monosugar or disugar moiety and 1-3 substituent (Y) on the distal 2-14 carbons from the distal end of the monosugar or disugar, wherein each Y is independently C₁₋₈ linear alkyl, C₃₋₈ branched alkyl, C₃₋₈ cycloalkyl, halogen, hydroxyl, monocyclic aromatic, monocyclic heteroaromatic, bicyclic aromatic, bicyclic heteroaromatic, tricyclic aromatic, or tricyclic heteroaromatic, wherein each Y is independently optionally substituted with C₁₋₈ linear alkyl, C₃₋₈ branched alkyl, C₃₋₈ cycloalkyl, halogen or hydroxyl.

In some embodiments, the present invention is directed to pharmaceutical compositions comprising a compound of Formula I.

In some embodiments, the present invention is directed to a method for treating a condition comprising administering to a patient in need thereof a therapeutically effective amount of a compound of Formula I or a pharmaceutical composition comprising a therapeutically effective amount of a compound of Formula I. Conditions that may be treated with the compounds or pharmaceutical composition disclosed herein may include, e.g., infections, burn wounds, cuts, cystic fibrosis, late stage illness, and combinations thereof.

The present invention is all directed to all variations, enantiomers and stereoisomers of the compounds disclosed herein.

### BRIEF DESCRIPTION OF THE DRAWINGS

The above and other features of the present disclosure, their nature, and various advantages will become more apparent upon consideration of the following detailed description, taken in conjunction with the accompanying drawings, in which:

FIG. 1 depicts the inhibition of PA PAO1 biofilm by the inventive compound 3,5-dimethyl-C12-βM and other compounds at 60 μM.

FIG. 2 depicts a general synthesis of 3,5-dimethyl-C12βM (A), examples of different bulky terminate groups (B) and chiral derivatization and resolution to obtain specific stereoisomers (C).

FIG. 3 depicts the results of Example 1.

FIG. 4 depicts the chemical structures of compounds used in Example 2

FIG. 5 depicts results of Example 2.

FIG. 6 depicts results of Example 2.

FIG. 7 depicts the results of Example 3.

FIG. 8 depicts the results of Example 4.

FIG. 9 depicts the results of Example 5.

FIG. 10 depicts the results of Example 6.

FIG. 11 depicts the results of Example 7.

FIG. 12 depicts the results of Example 8.

### DEFINITIONS

As used herein, the singular forms “a,” “an,” and “the” include plural references unless the context clearly indicates otherwise. Thus, for example, reference to “an excipient” includes a single excipient as well as a mixture of two or more different excipients, and the like.

As used herein, the term “about” in connection with a measured quantity, refers to the normal variations in that measured quantity, as expected by one of ordinary skill in the art in making the measurement and exercising a level of care commensurate with the objective of measurement and the precision of the measuring equipment. In certain embodiments, the term “about” includes the recited number±10%, such that “about 10” would include from 9 to 11.

As used herein, the term “active agent” refers to any material that is intended to produce a therapeutic, prophylactic, or other intended effect, whether or not approved by a government agency for that purpose. These terms with respect to specific agents include all pharmaceutically active agents, all pharmaceutically acceptable salts thereof, complexes, stereoisomers, crystalline forms, co-crystals, ether, esters, hydrates, solvates, and mixtures thereof, where the form is pharmaceutically active.

As used herein, the term “variations” refers to a compound's various isomers thereof, various structural modifications thereof, and combinations thereof.

As used herein, the term “stereoisomers” is a general term for all isomers of individual molecules that differ only in the orientation of their atoms in space. It includes enantiomers and isomers of compounds with one or more chiral centers that are not mirror images of one another (diastereomers).

The term “enantiomer” or “enantiomeric” refers to a molecule that is nonsuperimposable on its mirror image and hence optically active wherein the enantiomer rotates the plane of polarized light in one direction by a certain degree, and its mirror image rotates the plane of polarized light by the same degree but in the opposite direction.

The term “chiral center” refers to a carbon atom to which four different groups are attached.

The term “patient” refers to a subject, an animal or a human, who has presented a clinical manifestation of a particular symptom or symptoms suggesting the need for treatment, who is treated preventatively or prophylactically for a condition, or who has been diagnosed with a condition to be treated. The term “subject” is inclusive of the definition of the term “patient” and does not exclude individuals who are otherwise healthy.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to illuminate certain materials and methods and does not pose a limitation on scope. No language in the specification should be construed as indicating any non-claimed element as essential to the practice of the disclosed materials and methods.

The term “condition” or “conditions” refers to those medical conditions that can be treated or prevented by administration to a subject of an effective amount of the antibiotic pharmaceutical composition disclosed herein, e.g., infections, burn wounds, cuts, cystic fibrosis, late stage illness, and the like.

The terms “treatment of” and “treating” includes the lessening of the severity of or cessation of a condition or lessening the severity of or cessation of symptoms of a condition.

The terms “prevention of” and “preventing” includes the avoidance of the onset of a condition.

“Therapeutically effective amount” is intended to include an amount of an active agent, or an amount of the combination of active agents, to treat or prevent the condition, or to treat the symptoms of the condition, in a subject.

“Sub-lethal amount” is intended to include an amount of an active agent, or an amount of the combination of active agents, that is less than a therapeutically effective amount needed to treat or prevent a condition, or to treat or prevent the symptoms of a condition.

The phrase “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio.

For the purpose of the present disclosure, the term “aryl” or “aromatic” as used by itself or as part of another group refers to a monocyclic or bicyclic aromatic ring system having from six to fourteen carbon atoms (i.e., C₆₋₁₄ aryl) and also refers to tricyclic ring systems. Non-limiting exemplary aryl groups include phenyl (abbreviated as “Ph”), naphthyl, phenanthryl, anthracyl, indenyl, azulenyl, biphenyl, biphenylenyl, and fluorenyl groups. In one embodiment, the aryl group is chosen from phenyl or naphthyl.

For the purpose of the present disclosure, the term “heteroaryl” or “heteroaromatic” refers to monocyclic and bicyclic aromatic ring systems having 5 to 14 ring atoms (i.e., C₅₋₁₄ heteroaryl) and 1, 2, 3, or 4 heteroatoms independently chosen from oxygen, nitrogen and sulfur. The term also refers to tricyclic ring systems. In one embodiment, the heteroaryl has three heteroatoms. In another embodiment, the heteroaryl has two heteroatoms. In another embodiment, the heteroaryl has one heteroatom. In one embodiment, the heteroaryl is a C₅ heteroaryl. In another embodiment, the heteroaryl is a C₆ heteroaryl. Non-limiting exemplary heteroaryl groups include thienyl, benzo[b]thienyl, naphtho[2,3-b]thienyl, thianthrenyl, furyl, benzofuryl, pyranyl, isobenzofuranyl, benzooxazonyl, chromenyl, xanthenyl, 2H-pyrrolyl, pyrrolyl, imidazolyl, pyrazolyl, pyridyl, pyrazinyl, pyrimidinyl, pyridazinyl, isoindolyl, 3H-indolyl, indolyl, indazolyl, purinyl, isoquinolyl, quinolyl, phthalazinyl, naphthyridinyl, cinnolinyl, quinazolinyl, pteridinyl, 4aH-carbazolyl, carbazolyl, β-carbolinyl, phenanthridinyl, acridinyl, pyrimidinyl, phenanthrolinyl, phenazinyl, thiazolyl, isothiazolyl, phenothiazolyl, isoxazolyl, furazanyl, and phenoxazinyl. In one embodiment, the heteroaryl is chosen from thienyl (e.g., thien-2-yl and thien-3-yl), furyl (e.g., 2-furyl and 3-furyl), pyrrolyl (e.g., 1H-pyrrol-2-yl and 1H-pyrrol-3-yl), imidazolyl (e.g., 2H-imidazol-2-yl and 2H-imidazol-4-yl), pyrazolyl (e.g., 1H-pyrazol-3-yl, 1H-pyrazol-4-yl, and 1H-pyrazol-5-yl), pyridyl (e.g., pyridin-2-yl, pyridin-3-yl, and pyridin-4-yl), pyrimidinyl (e.g., pyrimidin-2-yl, pyrimidin-4-yl, pyrimidin-5-yl, and pyrimidin-5-yl), thiazolyl (e.g., thiazol-2-yl, thiazol-4-yl, and thiazol-5-yl), isothiazolyl (e.g., isothiazol-3-yl, isothiazol-4-yl, and isothiazol-5-yl), oxazolyl (e.g., oxazol-2-yl, oxazol-4-yl, and oxazol-5-yl) and isoxazolyl (e.g., isoxazol-3-yl, isoxazol-4-yl, and isoxazol-5-yl). The term “heteroaryl” is also meant to include possible N-oxides. Exemplary N-oxides include pyridyl N-oxide and the like.

### DETAILED DESCRIPTION

In certain embodiments, the compounds disclosed herein inhibit swarming motility and/or biofilm formation by Pseudomonas aeruginosa. In contrast, at low concentrations, the antibiotic tobramycin, a front-line treatment for chronic infections associated with cystic fibrosis (CF), increases swarming motility and biofilm formation by Pseudomonas aeruginosa. In certain embodiments, the compounds disclosed herein may be used as front line antibiotic treatment or adjuvant agents to increase the effectiveness of antibiotics such as tobramycin. The compounds disclosed herein alone or in combination with antibiotics, eliminate or inhibit persisters in biofilm that are induced upon exposure to antibiotic treatment.

In certain embodiments, the present invention is directed to a compound of Formula I:

X—Z (I)

wherein X is a monosugar or disugar moiety and Z is a C₈₋₂₀ straight chain alkyl, alkenyl or alkynyl having 1-5 substituents (Y) on the first 6 carbons proximal to the monosugar or disugar moiety, wherein each Y is independently C₁₋₈ linear alkyl, C₃₋₈ branched alkyl, C₃₋₈ cycloalkyl, halogen, hydroxyl, monocyclic aromatic, monocyclic heteroaromatic, bicyclic aromatic, bicyclic heteroaromatic, tricyclic aromatic, or tricyclic heteroaromatic, wherein each Y is independently optionally substituted with C₁₋₈ linear alkyl, C₃₋₈ branched alkyl, C₃₋₈ cycloalkyl, halogen or hydroxyl. In such embodiments, there may be 1 substituent on any of carbon 1, 2, 3, 4, 5 or 6. In other embodiments, there may one substituent on carbon 1 and one substituent on either carbon 2, 3, 4, 5 or 6. In other embodiments, there may one substituent on carbon 2 and one substituent on either carbon 3, 4, 5 or 6. In other embodiments, there may one substituent on carbon 3 and one substituent on either carbon 4, 5 or 6. In other embodiments, there may one substituent on carbon 4 and one substituent on either carbon 5 or 6. In other embodiments, there may one substituent on carbon 5 and one substituent on carbon 6. In particular compounds, there is substitution on the 3 and 5 carbons proximal to the sugar moiety.

In certain embodiments, the disugar is selected from the group consisting of β-maltoside (βM), α-maltoside (αM), β-cellobioside (βC) and α-cellobioside (αC).

In certain embodiments, the Y is independently selected from the group consisting of methyl, ethyl, linear or branched propyl, and linear or branched butyl.

In certain embodiments, the compound is 3,5-dimethyl-C12-βM, 3,5-dimethyl-C12-αM, 3,5-dimethyl-C12-βC or 3,5-dimethyl-C12-αC.

In certain embodiment the compound has 1-4 substituents (Y) on the proximal 4 carbons to the monosugar or disugar moiety.

In certain embodiments, the present invention is directed to a compound of Formula

X—Z (I)

wherein X is a monosugar or disugar moiety and Z is a C₈₋₂₀ straight chain alkyl, alkenyl or alkynyl having 2-5 substituents (Y) on the first 6 carbons proximal to the monosugar or disugar moiety and 1-3 substituent (Y) on the distal 2-14 carbons from the distal end of the monosugar or disugar, wherein each Y is independently C₁₋₈ linear alkyl, C₃₋₈ branched alkyl, C₃₋₈ cycloalkyl, halogen, hydroxyl, monocyclic aromatic, monocyclic heteroaromatic, bicyclic aromatic, bicyclic heteroaromatic, tricyclic aromatic, or tricyclic heteroaromatic, wherein each Y is independently optionally substituted with C₁₋₈ linear alkyl, C₃₋₈ branched alkyl, C₃₋₈ cycloalkyl, halogen or hydroxyl.

In certain embodiments, the present invention is directed to a pharmaceutical composition comprising (i) a compound of Formula I as disclosed above in combination with a pharmaceutically acceptable excipient. The pharmaceutical composition can comprise the compound of the present invention in an amount (w/w) from about 1% to about 99%, about 10% to about 90%, about 20% to about 80%, about 30% to about 70%, about 40% to about 60% or about 45% to about 55%.

In certain embodiments, the pharmaceutical composition is suitable for oral, sublingual, topical, rectal, pulmonary, intranasal or parenteral administration

In certain embodiments, the pharmaceutical composition is suitable for topical administration wherein the composition is in a form selected from a solution, suspension, cream, ointment, gel or transdermal patch.

In certain embodiments, the pharmaceutical composition is suitable for pulmonary administration wherein the composition is a solution or suspension and is contained in a metered dose inhaler or nebulizer.

In certain embodiments, the pharmaceutical composition is suitable for pulmonary administration wherein the composition is a powder and is contained in a dry powder inhaler.

In certain embodiments, the present invention is directed to a method of treating a condition comprising administrating to a patient in need thereof a compound or pharmaceutical composition as disclosed herein.

In certain embodiments, the condition is selected from infection, burn wounds, cuts, cystic fibrosis, late stage illness or combinations thereof.

In certain embodiments, the method is comprises administration by a route selected from oral, sublingual, topical, rectal, pulmonary, intranasal or parenteral administration.

In certain embodiments, the present invention is directed to a method of preventing, inhibiting or reducing the formation of a biofilm comprising contacting the biofilm with a compound or pharmaceutical composition as disclosed herein. The contacting can be done in-vivo or in-vitro. In such embodiments, the biofilm can be formed by, e.g., Pseudomonas Aeruginosa.

In certain methods disclosed herein, there is a reduction of cycic-di-GMP in the bacteria in an infection condition or site. The reduction can be, e.g., greater than about 10%, greater than about 25%, greater than about 40%, greater than about 60%, greater than about 80% or greater than about 90%.

In certain methods disclosed herein, there is a prevention or reduction of the formation of drug (antibiotic)-tolerant bacteria during an antibiotic treatment of an infection condition or site. The reduction can be, e.g., greater than about 10%, greater than about 25%, greater than about 40%, greater than about 60%, greater than about 80% or greater than about 90%.

In certain methods disclosed herein, there is a prevention or reduction in the increase of persister bacteria during an antibiotic treatment of an infection condition or site. The reduction can be, e.g., greater than about 10%, greater than about 25%, greater than about 40%, greater than about 60%, greater than about 80% or greater than about 90%.

In certain methods disclosed herein, there is a prevention or reduction of gene transfer between bacteria that results in spreading of drug resistance among the bacteria.

For inhalation or intranasal administration, the agent can be administered using a nebulizer, inhaler, atomizer, aerosolizer, mister, dry powder inhaler, metered dose inhaler, metered dose sprayer, metered dose mister, metered dose atomizer, or other suitable delivery device.

The antibiotic pharmaceutical composition disclosed herein may be suitable for topical administration. For instance, the composition may be administered through a transdermal delivery device or may be in the form of a transdermal topical formulation such as an ointment, a cream, a gel, a topical solution, a lotion, a foam, a topical powder, or a paste.

In some embodiments, the antibiotic pharmaceutical composition may further comprise a pharmaceutically acceptable excipient. The excipient can be in an amount (w/w) from about 1% to about 99%, about 10% to about 90%, about 20% to about 80%, about 30% to about 70%, about 40% to about 60% or about 45% to about 55%.

The pharmaceutically acceptable excipient may include, without limitations, solvents, suspension mediums, surfactants (e.g., dodecyl b-maltoside), dyes, perfumes, thickening agents, stabilizers, skin penetration enhancers, preservatives, antioxidants, other active agents (e.g., anesthetics or analgesics) and combinations thereof.

For topical formulations, the antibiotic pharmaceutical composition can optionally include one or more penetration enhancers, which increase the rate at which the active agent(s) in the composition penetrate through the patient's skin. Suitable penetration enhancers include, but are not limited to, C₂-C₄ alcohols such as ethanol and isopropanol, polyethylene glycol monolaurate, polyethylene glycol-3-lauramide, dimethyl lauramide, sorbitan trioleate, fatty acids, esters of fatty acids having from about 10 to about 20 carbon atoms, monoglycerides or mixtures of monoglycerides of fatty acids having a total monoesters content of at least 51% where the monoesters are those with from 10 to 20 carbon atoms, and mixtures of mono-, di- and tri-glycerides of fatty acids. Suitable fatty acids include, but are not limited to lauric acid, myristic acid, stearic acid, oleic acid, linoleic acid and palmitic acid. Monoglyceride permeation enhancers include glycerol monooleate, glycerol monolaurate, and glycerol monolinoleate, for example.

The antibiotic pharmaceutical composition may optionally include one or more preservatives, e.g., methyl hydroxybenzoate, propyl hydroxybenzoate, chlorocresol, benzalkonium chlorides, and the like.

Other suitable excipients may include, for example, starch, glucose, lactose, mannitol, magnesium stearate, talc, cellulose, magnesium carbonate, sodium bicarbonate, citric acid, water, saline solution, aqueous dextrose, glycerol, alcohols (e.g., propylene glycol, phenoxyethanol, methanol, ethanol, isopropyl alcohol, and mixtures thereof) mineral oil, lanolin, gums of vegetable origin, polyalkylene glycols, and the like.

Surfactants useful in the compositions of the present invention include those selected from the group consisting of dodecyl b-maltoside, sarcosinates, dioctyl sodium sulfoscuccinate, pluronic F68, sodium lauryl sulfate, sorbitan monolaurate, lauryldimethylamineoxide, lauric-diethanolamide, PEG-Esters (polyethylene glycol-dilaurate), coconut hydroxyethyl imidazoline, sodium sulfosuccinate ester of lauric MEA, sodium sulfosuccinate ester of ethoxylated lauryl alcohol, lauric-monoethanolamide, bis-(2-hydroxyethyl) cocoamine oxide, polyoxypropylene bases, coconut fatty acid, 2-sulfo-ester, sodium salt, N-coconut oil acyl-N-methyl taurine, sodium salt, lauroyl sarcosine, 30% sodium lauryl sarcosinate, sodium lauroyl sarcosinate, myristoyl sarcosine, oleoyl sarcosine, stearoyl sarcosine, polyoxyethelene 21 stearyl ether (0.1 BHA & 0.005% citric acid as preservatives), lauroamphoglycinate, lauroamphocarboxyglycinate, lauroamphocarboxypropinate, lauroamphocarboxyglycinate-sulfanate, sodium lauryl sulfate (66% lauryl, 27% myristyl, 71% cetyl), polyoxyethylene sorbitan mono-oleate, and mixtures thereof.

### Methods of Treatment

In some embodiments, the present invention is directed to a method of treating a condition, such as an infection, a burn wound, cuts, cystic fibrosis, late stage illness or a combination thereof. The method may comprise administering or applying an antibiotic pharmaceutical composition comprising a compound disclosed herein and optionally in combination with another antibiotiv, according to any of the embodiments disclosed herein to a patient in need thereof.

In some embodiments, administering or applying the antibiotic pharmaceutical composition inhibits bacterial motility.

### Additional Antibiotics

In certain embodiments, the present invention is directed to pharmaceutical formulations comprising one or more of the adjuvants disclosed herein in combination with an antibiotic. Other embodiments are directed to combination therapy for treating infections comprising administering one or more of the adjuvants disclosed herein with an antibiotic. The adjuvant can be in the same formulation or a different formulation than the antibiotic. The adjuvant can also be administered by a different route (e.g., oral, nasal, parenteral, inhalation, topical) than the antibiotic. The administration can be before, concurrently or after the administration of the antibiotic.

The term “antibiotic” is used to refer to antibacterial agents that may be derived from bacterial sources. Antibiotic agents may be bactericidal and/or bacteriostatic.

The antibiotic used in combination with the compounds of the present invention may be aminoglycosides, ansamycins, carbacephem, carbapenems, cephalosporins (including first, second, third, fourth and fifth generation cephalosporins), lincosamides, macrolides, monobactams, nitrofurans, quinolones, penicillin, sulfonamides, polypeptides and tetracycline.

In certain embodiments, the antibiotic may be an aminoglycoside such as Amikacin, Gentamicin, Kanamycin, Neomycin, Netilmicin, Tobramycin or Paromomycin.

In other embodiments, the antibiotic agent may be a carbapenem such as Ertapenem, Doripenem, Imipenem/Cilastatin or Meropenem.

In further embodiments, the antibiotic agent may be a cephalosporin (first generation) such as Cefadroxil, Cefazolin, Cefalexin, Cefalotin or Cefalothin, or alternatively a Cephalosporin (second generation) such as Cefaclor, Cefamandole, Cefoxitin, Cefprozil or Cefuroxime. Alternatively the antibiotic agent may be a Cephalosporin (third generation) such as Cefixime, Cefdinir, Cefditoren, Cefoperazone, Cefotaxime, Cefpodoxime, Ceftibuten, Ceftizoxime and Ceftriaxone or a Cephalosporin (fourth generation) such as Cefepime and Ceftobiprole.

In other embodiments, the antibiotic agent may be a lincosamides such as Clindamycin and Azithromycin, or a macrolide such as Azithromycin, Clarithromycin, Dirithromycin, Erythromycin, Roxithromycin, Troleandomycin, Telithromycin and Spectinomycin.

In further embodiments, the antibiotic agent may be a monobactams such as Aztreonam, or a nitrofuran such as Furazolidone or Nitrofurantoin.

In other embodiments, the antibiotic agent may be a penicillin such as Amoxicillin, Ampicillin, Azlocillin, Carbenicillin, Cloxacillin, Dicloxacillin, Flucloxacillin, Mezlocillin, Nafcillin, Oxacillin, Penicillin G or V, Piperacillin, Temocillin and Ticarcillin.

In further embodiments, the antibiotic agent may be a sulfonamide such as Mafenide, Sulfonamidochrysoidine, Sulfacetamide, Sulfadiazine, Silver sulfadiazine, Sulfamethizole, Sulfamethoxazole, Sulfanilimide, Sulfasalazine, Sulfisoxazole, Trimethoprim, and Trimethoprim-Sulfamethoxazole (Co-trimoxazole) (TMP-SMX).

In further embodiments, the antibiotic agent may be a quinolone such as Ciprofloxacin, Enoxacin, Gatifloxacin, Levofloxacin, Lomefloxacin, Moxifloxacin, Nalidixic acid, Norfloxacin, Ofloxacin, Trovafloxacin, Grepafloxacin, Sparfloxacin and Temafloxacin.

In other embodiments, the antibiotic agent may be a polypeptide such as Bacitracin, Colistin and Polymyxin B or a tetracycline such as Demeclocycline, Doxycycline, Minocycline and Oxytetracycline

In particular the antibiotic agent may be tobramycin, colistin, neomycin or polymixin B.

In certain embodiments, the antibiotic agent is active in the treatment or prophylaxis of infections caused by gram-negative or gram-positive bacteria, such as Escherichia coli and Klebsiella particularly Pseudomonas aeruginosa.

The following examples are set forth to assist in understanding the invention and should not, of course, be construed as specifically limiting the invention described and claimed herein. Such variations of the invention, including the substitution of all equivalents now known or later developed, which would be within the purview of those skilled in the art, and changes in formulation or minor changes in experimental design, are to be considered to fall within the scope of the invention incorporated herein.

### EXAMPLES

### General Procedures

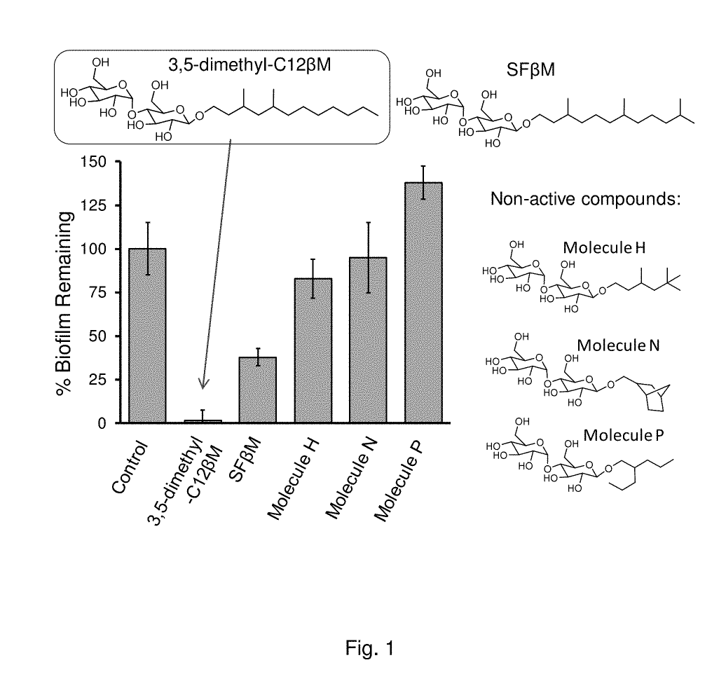

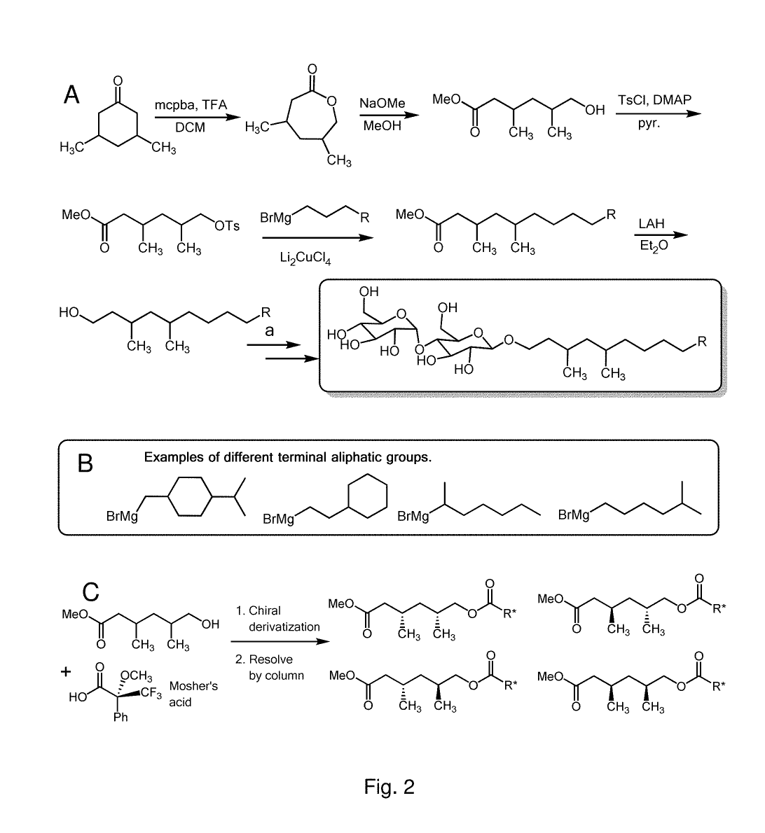

Our inventive example, 3,5-dimetyl-C12-βM, is more active at inhibiting biofilms than the comparative compound, SUM (FIG. 1). The molecule 3,5-dimetyl-C12-βM consists of a disugar tethered with a C12-chain having two methyl substitutions at the 3,5 position, whereas SUM has three methyl substitutions at the 3,7,11 positions (the saturated farnesol moiety) on the hydrocarbon chain (FIG. 1). This change of methyl substitution position unexpectedly increased the potency of biofilm inhibition from ˜60% by SFβM to >95% (FIG. 1). This result suggests that bulky structural features close to the sugar moiety can modulate activity. The synthesis of 3,5-dimethyl-C12-βM is shown in FIG. 2A. Different end groups may be incorporated using different Grignard reagents (RMgBr) to conduct the alkylation (FIG. 2B). The 3,5-dimethyl substituent on the hydrocarbon chain belongs to a class of pheromone structures, for which the stereochemistry is well studied. We will use chiral derivatization to facilitate the resolution to obtain each specific stereomer (FIG. 2C). This approach directly incorporates a synthetic scheme that has been validated. Since the chiral Mosher's acid for chiral derivatization is close to the methyl substitution (3 atoms away), we expect that some diastereomers will form more readily than others, and that their separation on a normal prep-TLC is possible. For exemplification, four stereoisomers of the 3,5-dimethyl groups, four different end group substitutions, two different disugars (maltoside and cellubioside), and two glycosidic bonds (α- and β-) may be synthesized.

### Example 1

The following procedure was performed in order to show the effects of 3,5-DβM and 3,5-DβC to persister population during Biofilm Formation.

1. Grow 24 h biofilm without tobramycon with and without agent (3,5-DβM or 3,5-DβC).

2. Sonicate biofilm on pegs in saline to obtain bacteria inside biofilm.

3. Treat obtained bacteria with 20 μg/ml tobrmaycin for 8 h to isolate persister.

4. Plate to count CFU/ml and normalized by biomass quantified by CV dye.

The same procedure is then performed with tobramycin.

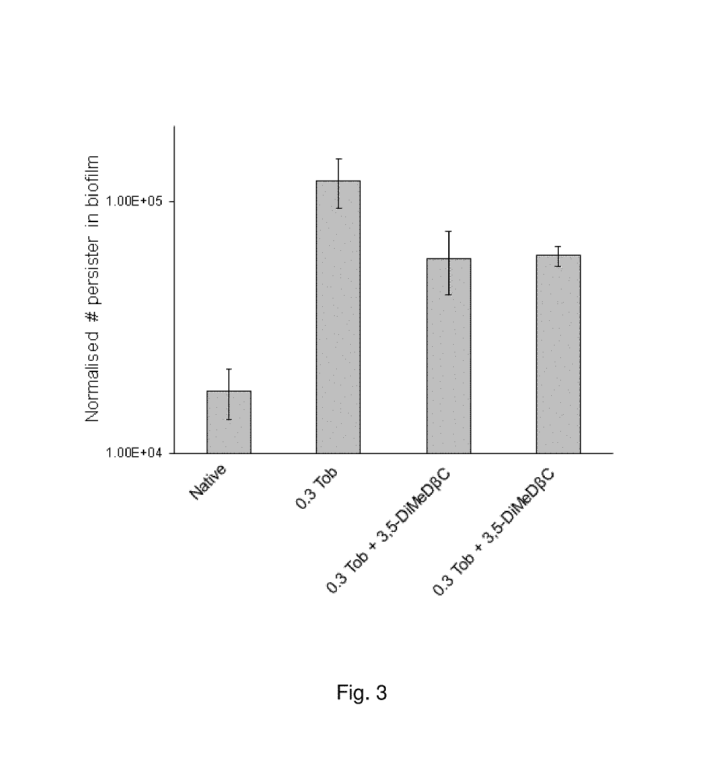

The results are depicted in FIG. 3.

### Example 2

Inventive examples 3,5-DβM and 3,5-DβC were compared to comparative biofilm inhibitors as follows.

| Biofilm Inhibitor Agents | Known Function | Consc. used in this assay | |
|---|---|---|---|
| Sodium Nitroprusside | cdG modulator | 25 nM | (DC₅₀ = 50 nM) |
| (SNP) | | | |
| 2-ABI | QS inhibitor | 40 μM | (IC₅₀ = 47 μM) |

| | | |
|---|---|---|
| BF8 | QS inhibitor | 40 μM |
| 3,5-DiMeDβM | Pili & LecA | 40 μM |
| | inhibitor | |
| 3,5-DiMeDβC | Pili & LecA | 40 μM |
| | inhibitor | |

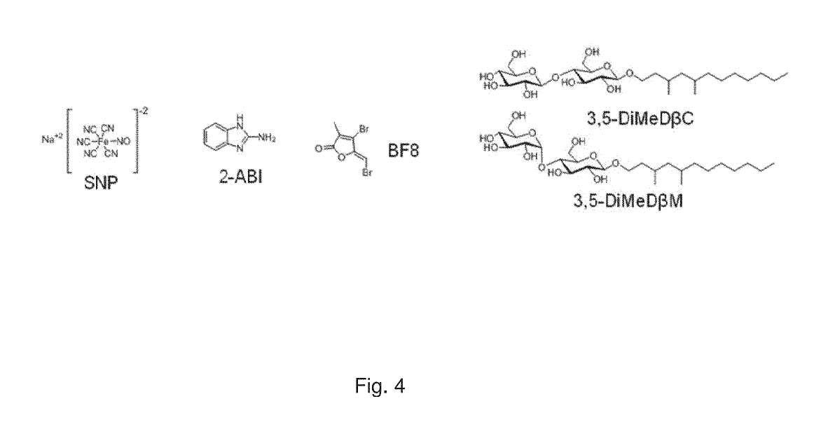

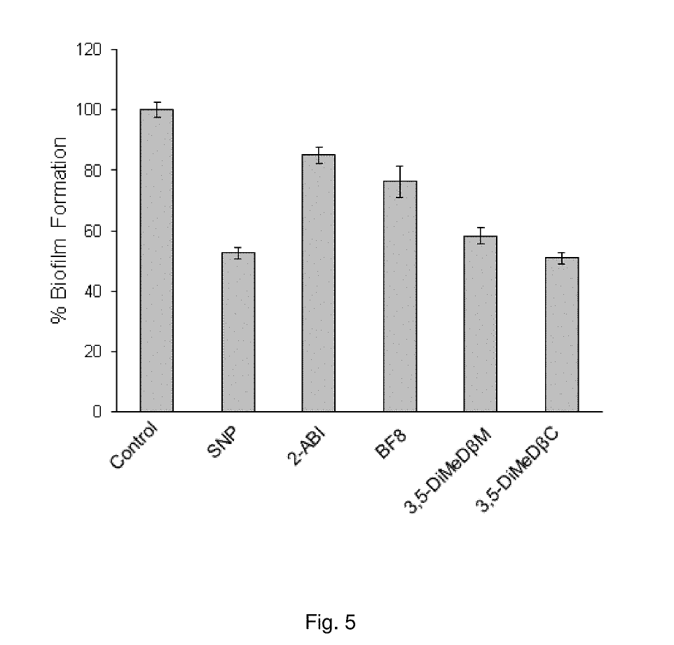

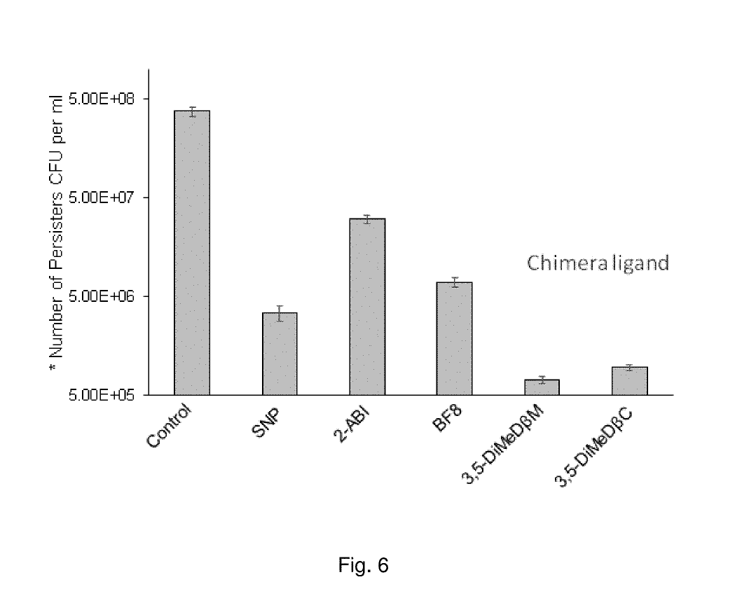

The chemical structures if each inhibitor are depicted in FIG. 4 and the following procedure was used for the assay.
1. The overnight PAO1 culture was diluted to OD˜0.01 in MHB medium and inoculated in MBEC plates with and without different biofilm inhibitors in the presence of 0.3 μg/ml Tobramycin for 24-h at static conditions.
2. The pegs were washed with PBS buffer 2 times and then sonicated to obtain bacteria inside biofilm in saline water.
3. The bacterial culture so obtained was centrifuged to obtain bacterial pellet.
4. The bacterial pellet is re-dispersed in 5 ml saline water and subjected to 20 μg/ml Tobramycin for 6-h to isolate persister.
5. The number of persister were calculated by colony forming units on LB agar plates for 24-h.
   The results are depicted in FIGS. 5 and 6. The results suggest the following.

### Example 3

Inventive examples 3,5-DβM and 3,5-DβC with tobramycin were tested according to the following procedure to show persister formation as compared to tobramycin alone.
1. The overnight PAO1 culture was diluted to OD˜0.01 in MHB medium and inoculated in MBEC plates in the presence of 0.3 μg/ml Tobramycin for 24-h at static conditions.
2. The pegs were washed with PBS buffer 2 times and treated with MHB containing 50 tobramycin with and without agents for 24-h.
3. Peg were washed twice and then sonicated to obtain bacteria inside biofilm in saline water.
4. The bacterial culture so obtained was centrifuged to obtain bacterial pellet.
5. The bacterial pellet is re-dispersed in 5 ml saline water and subjected to 20 μg/ml Tobramycin for 6-h to isolate persister.
6. The number of persister were calculated by colony forming units on LB agar plates for 24-h.

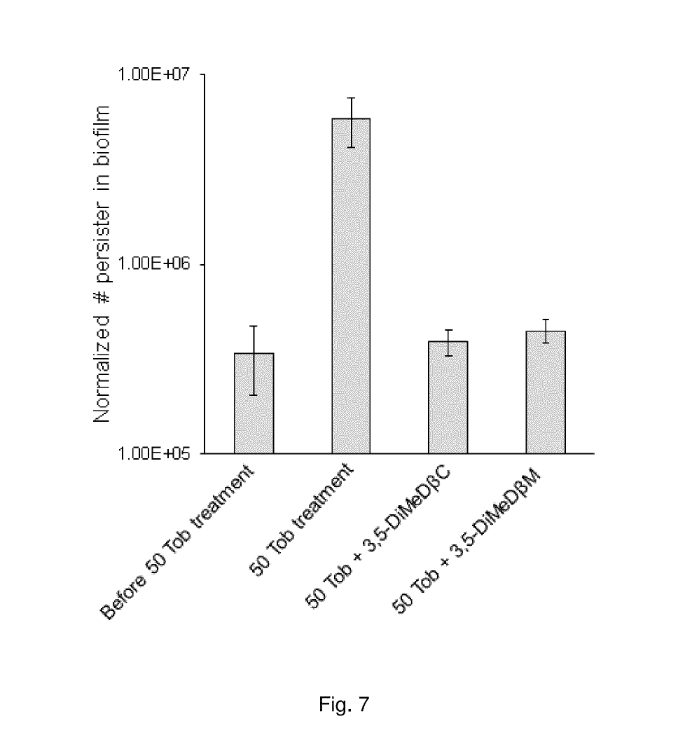

The results are shown in FIG. 7.

### Example 4

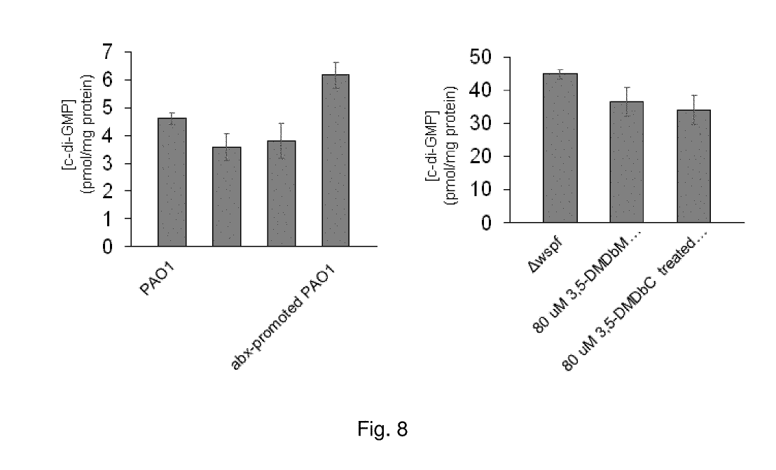

PAO1 in residual biofilm dispersed by 3,5-DiMeDβM and 3,5-DiMeDβC was tested by Liquid Chromatography-Mass Spectrometry (LC-MS). The results as depicted in FIG. 8 show that there is lower cdG than in original native or abx-promoted biofilm.

### Example 5

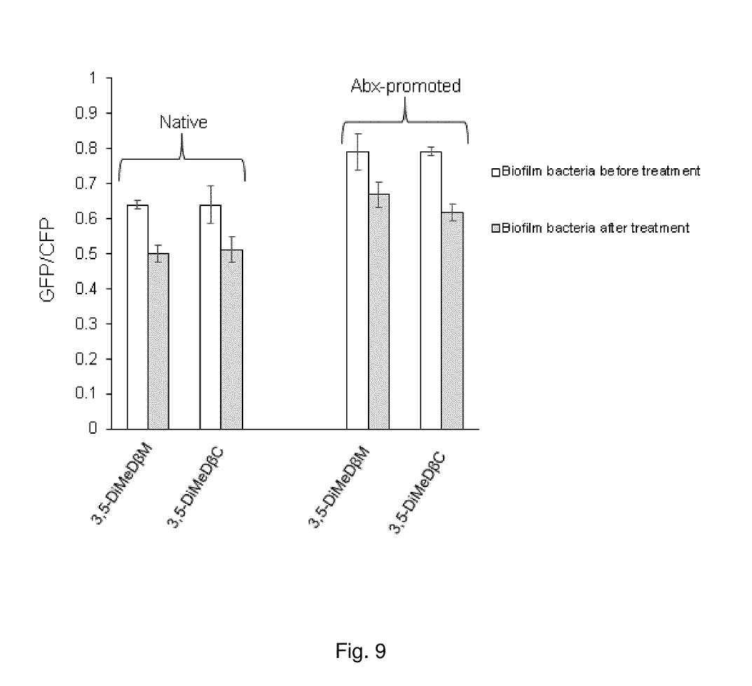

PAO1 in residual biofilm dispersed by 3,5-DiMeDβM and 3,5-DiMeDβC was tested by reporter strains and quantifying fluorescence according to the following procedure.
1. The PAO1 biofilms (native and 0.3-T promoted) were grown on peg microplate for 24-h in M63 medium.
2. To generate dispersed cells, the biofilms on pegs were washed twice with 0.9% NaCl and resuspended in M9 medium containing 3,5-DiMeDβM/C (40 μM for native and 85 μM for abx-promoted).
3. Biofilms were dispersed for 5-h at 37° C.
4. Biofilm cells were obtained by sonication in 0.9% NaCl.
5. An emission bandwidth of 440-503 nm was used for CFP, and 497-598 nm was used for GFP.
6. The relative fluorescence, GFP/CFP was calculated and was used as a measure of c-di-GMP per cell.
7. Note: CFP related # of bacteria while GFP related to cdG levels.
   The results as depicted in FIG. 9 show that there is lower cdG than in original native or abx-promoted biofilm.

### Example 6

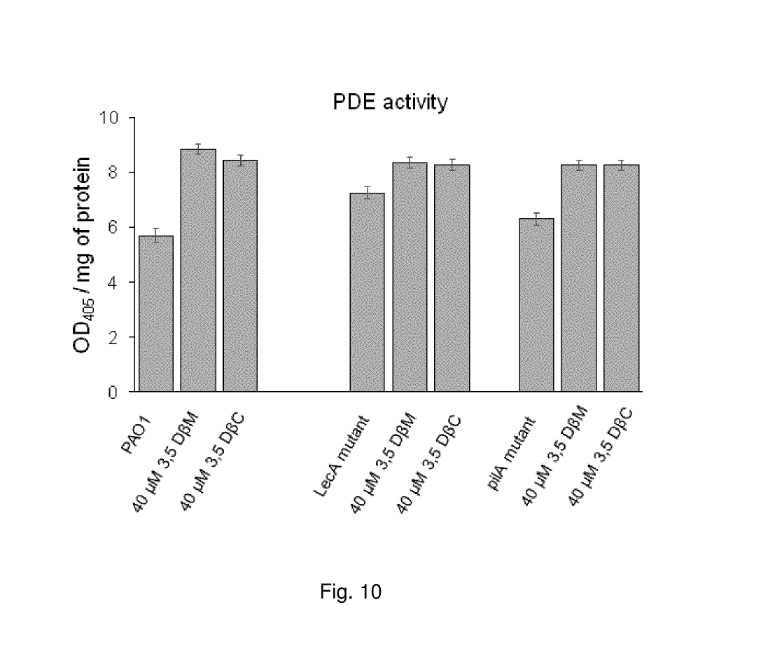

The effect of 3,5-DiMeDβM and 3,5-DiMeDβC on phosphodiesterase activity in biofilms of PA strains was tested according to the following procedure.
1. Bacteria are incubated with and without agents for 24-h to form biofilm;
2. Bacterial cells inside biofilm are lysed and PDE activity is tested by bis-NPP.
3. Protein is quantified by Bradford assay.
   As depicted in FIG. 10, the inventive compounds increase phosphodiesterase activity which is believed to result in decreased biofilm formation.

### Example 7

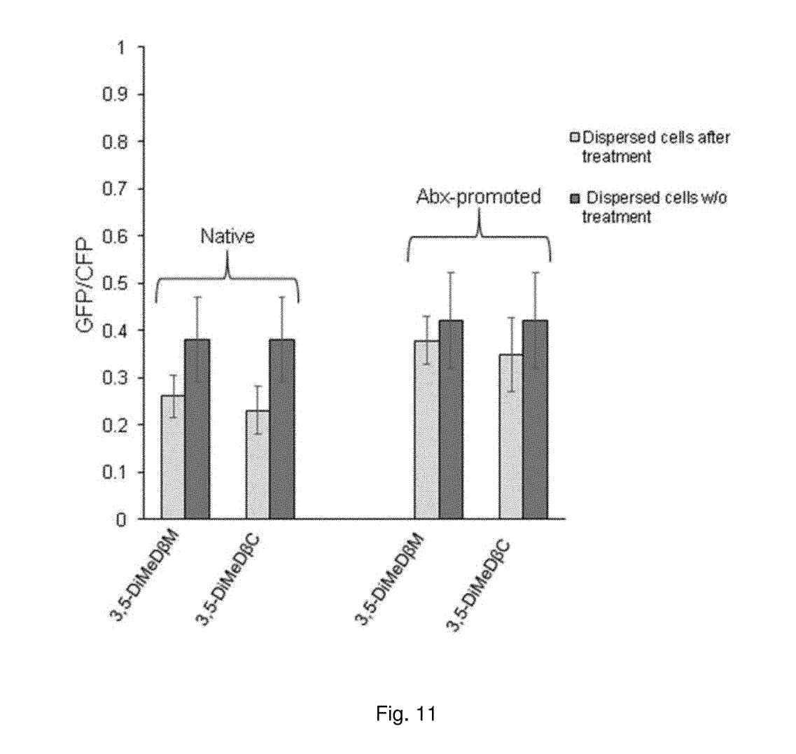

PAO1 in residual biofilm dispersed by 3,5-DiMeDβM and 3,5-DiMeDβC was tested by reporter strains. The results as depicted in FIG. 11 show that there is lower cdG than in naturally dispersed PAO1.

### Example 8

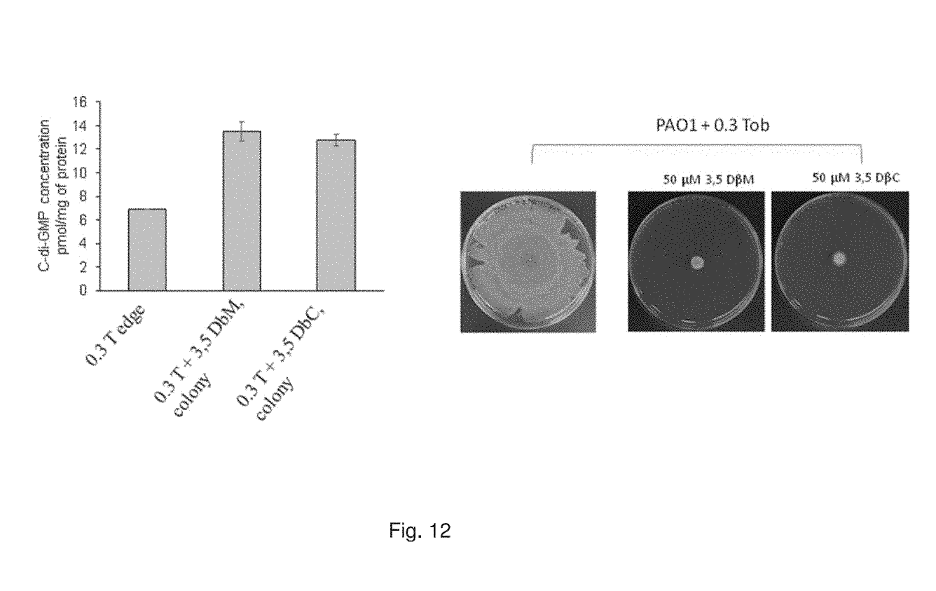

3,5-DiMeDβM and 3,5-DiMeDβC was tested by Liquid Chromatography-Mass Spectrometry (LC-MS) to show the effect on c-di-GMP on abx-promoted swarming P. aeruginosa. The results as depicted in FIG. 12 show that an increase on The results as depicted in FIG. 12 show that 3,5-DiMeDβM and 3,5-DiMeDβC increases c-di-GMP on abx-promoted swarming P. aeruginosa

For simplicity of explanation, the embodiments of the methods of this disclosure are depicted and described as a series of acts. However, acts in accordance with this disclosure can occur in various orders and/or concurrently, and with other acts not presented and described herein. Furthermore, not all illustrated acts may be required to implement the methods in accordance with the disclosed subject matter. In addition, those skilled in the art will understand and appreciate that the methods could alternatively be represented as a series of interrelated states via a state diagram or events.

In the foregoing description, numerous specific details are set forth, such as specific materials, dimensions, processes parameters, etc., to provide a thorough understanding of the present invention. The particular features, structures, materials, or characteristics may be combined in any suitable manner in one or more embodiments. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. Reference throughout this specification to “an embodiment”, “certain embodiments”, or “one embodiment” means that a particular feature, structure, or characteristic described in connection with the embodiment is included in at least one embodiment. Thus, the appearances of the phrase “an embodiment”, “certain embodiments”, or “one embodiment” in various places throughout this specification are not necessarily all referring to the same embodiment.

The present invention has been described with reference to specific exemplary embodiments thereof. The specification and drawings are, accordingly, to be regarded in an illustrative rather than a restrictive sense. Various modifications of the invention in addition to those shown and described herein will become apparent to those skilled in the art and are intended to fall within the scope of the appended claims.

What is claimed is:

1. A compound of Formula I:
X—Z (I)
wherein X is a monosugar or disugar moiety and Z is a C₈₋₂₀ straight chain alkyl, alkenyl or alkynyl having 1-5 substituents (Y) on the first 6 carbons proximal to the monosugar or disugar moiety, wherein each Y is independently C₁₋₈ linear alkyl, C₃₋₈ branched alkyl, C₃₋₈ cycloalkyl, halogen, hydroxyl, monocyclic aromatic, monocyclic heteroaromatic, bicyclic aromatic, bicyclic heteroaromatic, tricyclic aromatic, or tricyclic heteroaromatic, wherein each Y is independently optionally substituted with C₁₋₈ linear alkyl, C₃₋₈ branched alkyl, C₃₋₈ cycloalkyl, halogen or hydroxyl.

2. The compound of claim 1, wherein the disugar is selected from the group consisting of β-maltoside (βM), α-maltoside (αM), β-cellobioside (βC) and α-cellobioside (αC).

3. The compound of claim 1, wherein Y is independently selected from the group consisting of methyl, ethyl, linear or branched propyl, and linear or branched butyl.

4. The compound of claim 1, which is 3,5-dimethyl-C12-βM, 3,5-dimethyl-C12-αM, 3,5-dimethyl-C12-βC, 3,5-dimethyl-C12-αC.

5. The compound of claim 1, having 1-4 substituents (Y) on the proximal 4 carbons to the monosugar or disugar moiety.

6. A pharmaceutical composition comprising (i) a compound of Formula I:
X—Z (I)
wherein X is a monosugar or disugar moiety and Z is a C₈₋₂₀ straight chain alkyl, alkenyl or alkynyl having 1-5 substituents (Y) on the first 6 carbons proximal to the monosugar or disugar moiety, wherein each Y is independently C₁₋₈ linear alkyl, C₃₋₈ branched alkyl, C₃₋₈ cycloalkyl, halogen, hydroxyl, monocyclic aromatic, monocyclic heteroaromatic, bicyclic aromatic, bicyclic heteroaromatic, tricyclic aromatic, or tricyclic heteroaromatic, wherein each Y is independently optionally substituted with C₁₋₈ linear alkyl, C₃₋₈ branched alkyl, C₃₋₈ cycloalkyl, halogen or hydroxyl; and a pharmaceutically acceptable excipient.

7. The pharmaceutical composition of claim 6, wherein the disugar is selected from the group consisting of β-maltoside (βM), α-maltoside (αM), β-cellobioside (βC), α-cellobioside (αC) and rhamnose and the monosugar is galactose.

8. The pharmaceutical composition of claim 6, wherein Y is independently selected from the group consisting of methyl, ethyl, linear or branched propyl, and linear or branched butyl.

9. The pharmaceutical composition of claim 6, which is 3,5-dimethyl-C12-βM, 3,5-dimethyl-C12-αM, 3,5-dimethyl-C12-βC, 3,5-dimethyl-C12-αC or pharmaceutically acceptable salts thereof.

10. The pharmaceutical composition of claim 6, wherein having 1-4 substituents (Y) on the proximal 4 carbons to the monosugar or disugar moiety.

11. The pharmaceutical composition of claim 6, wherein the pharmaceutical composition is suitable for oral, sublingual, topical, rectal, pulmonary, intranasal or parenteral administration.

12. The pharmaceutical composition of claim 11, suitable for topical administration wherein the composition is in a form selected from a solution, suspension, cream, ointment, gel or transdermal patch.

13. The pharmaceutical composition of claim 11, suitable for pulmonary administration wherein the composition is solution or suspension and is contained in a metered dose inhaler or nebulizer.

14. The pharmaceutical composition of claim 11, suitable for pulmonary administration wherein the composition is a powder and is contained in a dry powder inhaler.

15. A method of treating a condition comprising administrating to a patient in need thereof a compound of Formula I:
X—Z (I)
wherein X is a monosugar or disugar moiety and Z is a C₈₋₂₀ straight chain alkyl, alkenyl or alkynyl having 1-5 substituents (Y) on the first 6 carbons proximal to the monosugar or disugar moiety, wherein each Y is independently C₁₋₈ linear alkyl, C₃₋₈ branched alkyl, C₃₋₈ cycloalkyl, halogen, hydroxyl, monocyclic aromatic, monocyclic heteroaromatic, bicyclic aromatic, bicyclic heteroaromatic, tricyclic aromatic, or tricyclic heteroaromatic, wherein each Y is independently optionally substituted with C₁₋₈ linear alkyl, C₃₋₈ branched alkyl, C₃₋₈ cycloalkyl, halogen or hydroxyl.

16. The method of claim 15, wherein the condition is selected from infection, burn wounds, cuts, cystic fibrosis, late stage illness or combinations thereof.

17. The method of claim 15, wherein the compound is administered by a route selected from oral, sublingual, topical, rectal, pulmonary, intranasal or parenteral administration.

18. The method of claim 15, wherein the compound is administered topically and is in a form selected from a solution, suspension, cream, ointment, gel or transdermal patch.

19. The method of claim 15, wherein the compound is administered by the pulmonary route by a metered dose inhaler or nebulizer and is in the form of a solution or suspension.

20. The method of claim 15, wherein the compound is administered by the pulmonary route by a dry powder inhaler and is in the form of a powder.

21. A method of preventing, inhibiting or reducing the formation of a biofilm comprising contacting the biofilm with a compound of Formula I:
X—Z (I)
wherein X is a monosugar or disugar moiety and Z is a C₈₋₂₀ straight chain alkyl, alkenyl or alkynyl having 1-5 substituents (Y) on the first 6 carbons proximal to the disugar moiety, wherein each Y is independently C₁₋₈ linear alkyl, C₃₋₈ branched alkyl, C₃₋₈ cycloalkyl, halogen, hydroxyl, monocyclic aromatic, monocyclic heteroaromatic, bicyclic aromatic, bicyclic heteroaromatic, tricyclic aromatic, or tricyclic heteroaromatic, wherein each Y is independently optionally substituted with C₁₋₈ linear alkyl, C₃₋₈ branched alkyl, C₃₋₈ cycloalkyl, halogen or hydroxyl.

22. The method of claim 21, wherein the contacting is in-vivo.

23. The method of claim 21, wherein the contacting is in-vitro.

24. The method of claim 21, wherein the biofilm is formed by Pseudomonas Aeruginosa.

25. The method of claim 21, that results in a reduction of cyclic-di-GMP in the bacteria in an infection condition or site.

26. The method of claim 21, that results in preventing the formation of drug (antibiotic)-tolerant bacteria during an antibiotic treatment of an infection condition or site.

27. The method of claim 21, that results in preventing the increase of persister bacteria during an antibiotic treatment of an infection condition or site.

28. The method of claim 21, that results in preventing the gene transfer between bacteria that results in spreading of drug resistance among bacteria.

29. The pharmaceutical composition of claim 6, further comprising an additional antibiotic.

30. The method of claim 15, further comprising administering an additional antibiotic.
